(19) United States
(12) Patent Application Publication
(10) Pub. No.: US 2010/0003247 A1
(43) Pub. Date: Jan. 7, 2010

(54) ASSAY FOR METASTATIC COLORECTAL CANCER

(75) Inventors: Emanuel F. Petricoin, III, Gainesville,, VA (US); Lance A. Liotta, Bethesda, MD (US); Mariaelena Pierobon, Manassas, VA (US); Valerie Calvert, Arlington, VA (US)

Correspondence Address: FOLEY AND LARDNER LLP;SUITE 500, 3000 K STREET NW, WASHINGTON, DC, 20007, US

(73) Assignees: George Mason Intellectual Properties, Inc.; Istitutjo Superiore Di Sanita

(21) Appl. No.: 12/446,910

(22) PCT Filed: Oct. 29, 2007
(86) PCT No.: PCT/US07/22790

§ 371 Date: Apr. 23, 2009

Related U.S. Application Data
(60) Provisional application No. 60/854,724, filed on Oct. 27, 2006.

(51) Int. Cl. A61K 39/395; C12Q 1/02; A61K 31/4545; A61K 31/496; A61K 31/404; A61K 31/517; A61K 31/34; A61K 31/415; A61P 35/00

(52) U.S. Cl. 424/133.1; 435/29; 514/318; 514/252.18; 514/252.19; 514/415; 514/266.1; 514/473; 514/406

(57) ABSTRACT

This invention relates, e.g., to a method for predicting the prognosis, the likelihood of metastasis in, or the desirability of administering an aggressive therapy to, a subject with colorectal cancer, comprising determining, in a sample from the subject, the level of phosphorylation compared to a positive and/or negative reference standard, of one or more of: (a) AKT (S473); (b) BAD (S112); (c) cABL (T735); (d) ERK (T42/44); (e) MARCKS (S152-156); (0 p38MAPK (T180-182): (g) STAT 1 (Y701 ); (h) PTEN (S380); (i) EGFR (Y992); (j) PAK 1/2 (S 1 19/204); or (k) PKC zeta/lambda (T410-403); or the total amount of (1) COX-2 protein; wherein if the level of phosphorylation of one or more of a-i or the total amount of COX-2 protein (1) is elevated compared to the negative reference standard, and/or if the level of phosphorylation of j or k is decreased compared to the positive reference standard, the subject has poor prognosis, is likely to undergo metastasis, and/or is a good candidate for aggressive therapy. Also described are methods for treating subjects likely to develop metastatic colorectal carcinoma, and pharmaceutical compositions and kits for implementing methods of the invention.

This application claims the benefit of the filing date of U.S. Provisional Application No. 60/854,724, filed Oct. 27, 2006, which is incorporated by reference herein in its entirety.

### BACKGROUND INFORMATION

Human tumors rely on defective protein-based cell signaling processes, driven by post-translational modifications such as protein phosphorylation, to grow, survive and metastasize. These signaling networks are also the targets for most of the current and planned molecular targeted inhibitors. An example is HERCEPTIN, a drug that can block the hyperactive Epiderimial Growth Factor (EGF) signaling system in breast cancer. Only patients that have this signaling pathway over-expressed and activated respond to the therapy. It is particularly important to be able to distinguish patients who harbor more aggressive forms of cancer, possibly with undetectable metastasis, from those who have more indolent forms of cancer that do not metastasize, or that do not metastasize as quickly. These two groups of patients generally have significant differences in outcome, reflecting the differences in aggressiveness and the propensity of the tumor to metastasize. A biomarker that could discriminate between the two groups of patients would be of great benefit.

Gene expression analysis (nucleic acids) has allowed investigators to derive prognostic signatures for outcome for certain cancers; however, these endpoints are limited to simple stratification only. The signature cannot tell the physician how to treat the non-responder group; it simply can be used to decide who will respond and who won't. Furthermore, the analysis of the many genes in gene expression analysis is complex, and generally involves the use of algorithms and extensive computer analysis. Also, gene expression analysis does not reflect the activated or functional state of the protein drug targets (does not correlate with the phosphorylation state of signal pathway proteins). By contrast, protein-signalling profiling can provide a prognostic signature and, importantly, can provide information on therapies for treating patients with metastatic cancer. This is because the proteomic portraits are constructed on the drug targets themselves.

### DESCRIPTION OF THE DRAWINGS

FIG. 1 is a bar graph showing phosphorylation levels of the noted phosphoprotein targets and the total amount of COX-2 protein, wherein the phosphorylation state or amount of COX-2 correlates with metastatic colorectal cancer.

### DESCRIPTION OF THE INVENTION

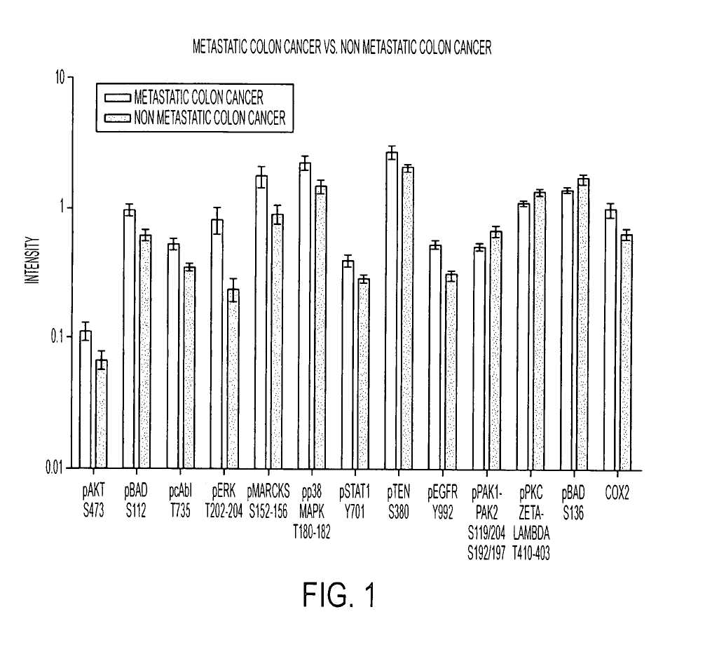

The present invention provides, e.g., combinations and methods for distinguishing between subjects having colorectal cancer (carcinoma) who are likely to develop metastatic cancer, and subjects who have a non-metastatic form of colorectal cancer. At least 12 protein markers are identified herein that exhibit an aberrant phosphorylation state (either over- or under-phosphorylated) and/or are overexpressed in subjects who have a metastatic form of colorectal cancer. See Example 1 and FIG. 1. The protein isoforms that exhibit an increased level of phosphorylation are activated; and the protein isoforms that exhibit a decreased level of phosphorylation are inactivated. COX-2 (another marker) is observed to be over-expressed in subjects presenting with metastatic cancer compared to subjects presenting with organ-confined primary colorectal cancer. Also discussed are methods for treating an aggressive phenotype of colorectal cancer (a phenotype that is associated with metastasis) based on the markers identified herein. An “aggressive phenotype,” as used herein, may include, e.g., one or more of the following: invasiveness through the full thickness of the bowel wall; spread to local regional lymph nodes; distant metastasis; short survival; or resistance to therapy. Colorectal cancers can metastasize to, e.g., lung and liver.

The methodology used to identify the markers of the invention was based on protein-signaling profiling. The observations presented herein provide the basis for a diagnostic assay (a prognostic signature, which serves to stratify patients), and identify new drug targets. This duality is sometimes referred to as a “theranostic”—wherein the measured analytes serve both as a diagnostic and a therapeutic target. Because a diagnostic assay of the invention requires the determination of the phosphorylation state of only a few proteins (or, in the case of COX-2, the total amount of the protein), the assay is simple to conduct and does not require complex, computer-based analysis. The treatment methods comprise inhibiting (suppressing, inactivating) the activated (over-phosphorylated) proteins with inhibitors, or activating (enhancing, stimulating) the under-phosphorylated proteins.

The invention relates, e.g., to a method for predicting whether a subject having colorectal cancer has a poor prognosis and/or has a form of colorectal cancer that is likely to metastasize, comprising determining, compared to a positive and/or a negative reference standard, in a sample from the subject, the level of phosphorylation of one or more of
a. pAKT (S473), and/or
b. pBAD (S112), and/or
c. pcABL (T735), and/or
d. pERK (T42/44), and/or
e. pMARCKS (S152-156), and/or
f. pp38MAPK (T180-182), and/or
g. pSTAT1 (Y701), and/or
h. pTEN (S380), and/or
i. pEGFR (Y992), and/or
j. pPAK1/2 (S119/204), and/or
k. pPKC zeta/lambda (T410-403), and/or
l. the total amount of COX-2 protein,
or a combination thereof,
wherein a significantly elevated level of phosphorylation of one or mole of a-i and/or of the total amount of COX-2 protein (1) compared to the negative reference standard, or a level that is statistically the same as the positive reference standard, and/or
a signiificantly reduced level ofpphosphorylation of one or more of j or k compared to the positive reference standard, or a level that is statistically the same as the negative reference standard,
indicates that subject has a poor prognosis and/or has a form of colorectal cancer that is likely to metastasize.

“Poor prognosis,” as used herein, includes a short period of being disease-free and/or short overall survival (less than 24 months overall survival). The term, a cancer is “likely to metastasize” means that the subject has greater than a 50% chance of developing metastasis.

In embodiments of the invention, any combination of two or more (e.g., 2, 3, 4, 5, 6, 7, 8, 9, 10, 11, or all 12) of the 12 markers noted above can be tested. For example, the level of phosphorylation and/or (in the case of COX-2) total amount of protein of the following subsets of markers can be tested:
(1) pAKT (S473), cABL (T735), pERK (T42/44), p38MAPK (T180-182), pEGFR (Y992), and/or COX2, including combinations thereof (e.g., combinations of 2, 4, or all 6 of the markers); or
(2) pAKT(S473), pBAD(S112) and pTEN (S380); or
(3) pEGFR (Y992) pAKT(S473), pBAD(S112) and pTEN (S380); or
(4) pERK (T42/44) and pp38MAPK (T180-182); or
(5) pEGFR (Y992) and pSTAT1 (Y701).

Embodiments of a method of the invention further comprise the following steps (all of which use conventional procedures that are well-known to skilled workers):
(a) obtaining a tissue specimen or biopsy from the subject;
(b) subjecting the tissue specimen or biopsy to laser capture microdissection in order to isolate epithelial cells;
(c) lysing the epitelial cells; and
(d) analyzing the lysate by an immunoassay to determine the phosphorylation state of the markers, (e.g., the level of phosphorylation of AKT(S473), cABL(T735), ERK(T42/44), p38MAPK(T180-192) and/or EGFR(Y992), and/or the total amount of COX-2) compared to the negative and/or positive reference standards. In one embodiment, the immunoassay is an ELISA. In another embodiment, the lysates are distributed as a suspension bead array or a reverse phase array and then subjected to an immunoassay; or the lysates are contacted with an array of antibodies or of aptamers, and are subjected to an immunoassay.

Another embodiment of the invention further comprises the following steps:
(a) obtaining a tissue specimen or biopsy from the subject; and
(b) analyzing the tissue specimen or biopsy by a histochemical method to determine if the phosphorylation state (e.g., level of phosphorylation of AKT(S473), cABL(T735), ERK(T42/44), p38MAPK(T180-192) and/or EGFR(Y992), and/or the total amount of COX-2) is significantly elevated compared to a control tissue specimen or biopsy. Suitable negative controls include, e.g., tissue or biopsy material obtained from a population of patients with indolent colorectal cancer; positive controls include, e.g., tissue or biopsy material obtained from a population of patients with aggressive colorectal cancer.

Surprisingly, although a wide variety of signaling pathways might have been expected to be correlated with the metastatic phenotype discussed herein, the up-regulated markers, (a)-(i) and (l), are members of a single, interconnected kinase signaling pathway, starting with the EGF receptor at the surface of a cell and ending with the nuclear transcriptional regulatory protein, COX-2. In one route of the pathway, EGFR and ABL phosphorylation leads to MARCKS phosphorylation, which in turn leads to ERK phosphorylation and p38 phosphorylation, which in turn leads to STAT1 phosphorylation. In the other route of the pathway, EGFR phosphorylation and ABL phosphorylation leads to PTEN phosphorylation, which in turn leads to AKT phosphorylation, which in turn leads to BAD phosphorylation and STAT1 phosphorylation. Ultimately, COX2 protein is then regulated transcriptionally by this pathway.

By contrast, members of many other signaling pathways do not show a significant correlation with the metastatic colorectal cancer phenotype. See, e.g., the proteins listed in Table 2 in Example 11.

A subject who has been determined by a method of the invention to have a poor prognosis, or to have a form of colorectal cancer which is likely to metastasize, is a good candidate for aggressive therapy and/or for treatment with targeted therapy.

By “aggressive therapy” is meant therapy that is designed to treat metastatic cancer and, preferably, is effective to ameliorate at least one or more of the effects of metastatic cancer. This can involve administering an agent (e.g. a drug) in an increased dosage or administering it more frequently than to a patient who is not a candidate for aggressive therapy, or selecting a therapy than is generally not given to a patient who is not a candidate for aggressive therapy (e.g. administering a more toxic fotm of chemotherapy). Other forms of aggressive therapy include radiation plus chemotherapy, and more aggressive surgery.

“Targeted therapy” refers to therapy with an agent (e.g., a drug) that is targeted against a particular target, such as one of the phosphoprotein targets identified herein.

For example, the targeted therapy can comprise the following:
if the subject exhibits a significantly elevated level of phosphorylation of AKT (S473), BAD (S112), cABL (T735), ERK (T42/44), MARCKS (S152-156), p38MAPK (T180-182), STAT1 (Y701), PTEN (S380), or EGFR (Y992), or a significantly elevated amount of COX-2 protein, the subject is treated with an effective amount of an inhibitor (e.g. an enzymatic inhibitor) of AKT, BAD cABL, ERK, MARCKS, p38MAPK, STAT1, PTEN, EGFR, or COX-2, respectively, and/or
if the subject exhibits a significantly reduced level of phosphiorylation of PAK1/2 (S119/204) or PKC zeta/lambda (T410-403), the subject is treated with an effective amount of an activator of PAK1/2 or PKC zeta/lambda, respectively.

An “effective amount,” as used herein, includes an amount that can bring about a detectable anti-metastatic effect.

The method of targeted therapy can comprise treatment with a combination of two or more of the inhibitors and/or activators. The cABL inhibitor can be, e.g., GLEEVEC, DASATINIB, and/or SUTENT; the EGFR inhibitor can be, e.g., TARCEVA, LAPATINIB, IRESSA, ERBITUX, and/or BEVTUZIMAB; and the COX-2 inhibitor can be, e.g., VIOXX and/or CELEBREX.

In one embodiment of the invention, if a subject exhibits a significantly elevated level of phosphorylation of EGFR(Y992) and/or ABL(T735) (e.g., exhibits the activation of the receptors, EGFR and/or ABL), the subject is treated with an effective amount of an inhibitor of a signaling kinase that lies downstream of these markers in the signaling pathway, such as an inhibitor of p38MAPK, and/or an inhibitor of AKT, and/or an inhibitor of ERK.

In another embodiment of the invention, if a subject exhibits a significantly elevated level of phosphorylation of AKT (S473), cABL (T735), ERK (T42/44), EGFR (Y992), or a significantly elevated amount of total COX-2, the subject is treated with a combination of an effective amount of carboxyamido imidazole (CAI) in combination with an AKT inhibitor, a cABL inhibitor, an ERK inhibitor, a COX-2 inhibitor, or an EGFR inhibitor, respectively.

In another embodiment of the invention, a subject is treated with a drug that is currently FDA approved, or is currently in Phase 2 or Phase 3 trials, albeit for other indications. It is noted that the present invention implicates these targets as being involved with colorectal cancer that is likely to present later with metastasis and thus has a much poorer prognosis, an indication that was not recognized previously. In this embodiment of the invention, if a subject exhibits a significantly elevated level of phosphorylation of AKT (S473), cABL (T735), ERK (T42/44), p38MAPK (T180-182), and/or EGFR (Y992), and/or of the total amount of COX-2 protein, the subject is treated, e.g., with an effective amount of the pAKT inhibitors, VQD-002 and/or Enzastaurin; the cABL inhibitors, GLEEVEC, SUTENT and/or DASATINIB; the ERK inhibitors, C1-1040 and/or PD0325901; the p38MAPK inhibitors, SCIO-469, SB 239063, VX-702, and/or BMS-582949; the pEGFR inhibitors, TARCEVA, LAPATINFB, IRESSA, ERBITUX and/or BEVTUZIMAB; or the COX-2 inhibitors, VIOXX and/or CELEBREX, respectively.

An inhibitor or activator can be targeted against one or more of the markers of the invention whose phosphorylation state is found to be aberrant (increased or decreased), and/or against COX-2. The inhibitor can be directed against the particular phosphorylated isoform of a protein analyzed in the Examples herein, or it can be directed against a different isoform, or against the phosphoprotein, in general. In one embodiment, the inhibitor(s) or activator(s) used in a treatment method are directed against a plurality of the targets (e.g. against 2, 3, 4, 5, 6, 7, 8, 9, 10, 11 or all 12 of the targets). One or more (e.g., 1, 2, 3, 4, 5 or more) inhibitor(s) or activator(s) may be used against any individual target. Suitable combinations of inhibitors include, e.g., two or more (e.g., 3, 4, 5, 6, 7, 8, 9 or 10) of VIOXX, CELEBREX, GLEEVEC, SUTENT, DASATINIB, TARCEVA, LAPATINIB, IRESSA, EPBITUX, BEVTUZIMAB.

As used herein, the singular forms “a,” “an” and “the” include plural referents unless the context clearly dictates otherwise. For example, “an” inhibitor, as used above, includes multiple inhibitors, e.g. 2, 3, 4, 5 or more inhibitors.

Another aspect of the invention is a method for treating a subject with colorectal carcinoma, comprising determining, compared to a positive and/or a negative reference standard, in a sample from the subject, the phosphorylation state of one or more of (a) pAKT (e.g., the level of phosphorylation of pAKT(S473)); (b) pBAD ((e.g., the level of phosphorylation of pBAD(S112)); (c) pcABL ((e.g., the level of phosphorylation of pcABL(T735)); (d) pERK ((e.g., the level of phosphorylation of pERK(T42/44)); (e) pMARCKS ((e.g., the level of phosphorylation of pMARCKS(S152-156)); (f) pp38MAPK ((e.g., the level of phosphorylation of pp38MAPK(T180-182)); (g) pSTAT1 ((e.g., the level of phosphorylation of pSTAT1(Y701)); (h) pTEN ((e.g., the level of phosphorylation of pTEN(S380)); (i) pEGFR ((e.g., the level of phosphorylation of pEGFR(Y992)); (j) pPAK1/2 ((e.g., the level of phosphorylation of pPAK1/2(S119/204)); and/or (k) pPKC zeta/lambda ((e.g., the level of phosphorylation of pPKCzeta/lambda(T410-403)); and/or (l) the total amount of COX-2 protein; including combinations thereof, and
if the phosphorylation state of one or more of (a)-(i) and/or the total amount of COX-2 protein (l) is significantly elevated compared to the negative reference standard, or at a level that is statistically the same as the positive reference standard, indicating that the subject has a poor prognosis and/or has a form of colorectal cancer that is likely to metastasize, the subject is administered an effective amount of an inhibitor of one or more of (a)-(i) (e.g. a kinase inhibitor or an enzyme inhibitor), or an inhibitor of COX-2, and/or
if the phosphorylation state of (j) or (k) is significantly decreased compared to the positive reference standard, or at a level that is statistically the same as the negative reference standard, indicating that the subject has a poor prognosis and/or has a form of colorectal cancer that is likely to metastasize, the subject is administered an effective amount of an activator (e.g. a kinase) of (j) and/or (k).

Combinations of these inhibitors and/or activators may be administered to the subject.

The “phosphorylation state” of a protein refers to the degree of (total amount of) phosphorylation of the protein. This includes both the number of sites (e.g. suitable Ser, Thr or Tyr amino acid residues) of the protein that are phosphorylated, and the level of phosphorylation at any given acceptor site on the amino acid chain. An increase in the phosphorylation state of a protein can reflect either an increase in the number of suitable amino acid residues of the protein (e.g., serines, threoninies or tyrosines) that are phosphorylated, or an increased frequency of phosphorylations at a particular amino acid residue. An “aberrant” phosphorylation state refers to a statistically significantly higher (elevated) or lower (decreased) phsophorylation state than a negative or positive reference standard, respectively.

A skilled worker will recognize that, in addition to the phosphorylated amino acid residues noted herein, a marker of the invention may be activated (or, in the case of pPAK1/2 or pPKC zeta/lambda, deactivated or inhibited) by the phosphorylation of other amino acid residues of the protein; and, in a method of the invention, the level of phosphorylation at one or more or those phosphorylated residues may be analyzed in addition to, or instead of, the noted residues. For example, for c-ABL, other sites include Y245, T735, and/or Y412; for EGFR, other sites include T669, S967,Y992, S1002, Y1045, S1046, S1057, Y1068, Y1086, Y1114, S1142, Y1148, and/or Y1173; for AKT, other sites include 473, 308 and/or T450; for pTEN, other sites include S380, T382 and/or T383; for STAT1, other sites include Y701 and/or S727; and for BAD, other sites include S155 and/or S112.

In an embodiment of the invention, a treatment method as discussed above may further comprise administering a conventional chemotherapeutic agent to the subject in combination with the inhibitor. As used herein, a “conventional chemotherapeutic agent” refers to a chemotherapeutic agent other than an inhibitor or activator of one of the 12 markers discussed herein. The conventional chemotherapeutic agent may be administered together with (concurrently with) the inhibitor or activator; or the agents may be administered sequentially.

In any of the methods described herein in which the level of phosphorylation of a particular phosphoprotein isoform is measured, the phosphorylation state of that protein (including, e.g., the level of phosphorylation of one or more of the other amino acid residues of the protein that contribute to its activation or, in the case of pPAK1/2 or pPKC zeta/lambda, to its inactivation) can be measured, instead.

Another aspect of the invention is in a method for treating colorectal cancel in a subject, the improvement comprising predicting by a method of the invention that the subject has a poor prognosis and/or has a form of colorectal cancer that is likely to later metastasize or later develop metastasis, and then treating the subject with a targeted therapy method of the invention.

A treatment method of the invention can inhibit and/or prevent metastasis of the colorectal cancer.

Another aspect of the invention is a collection of one or more agents suitable for assaying the phosphorylation state of one or more of the 11 phosphomarkers discussed herein, and/or the total amount of COX-2 protein, or a combination thereof. The agents may be specific for the particular phosphorylated isoforms indicated in Example 1, and/or they be specific for other phosphorylated amino acid residues of the proteins. The collection may contain agents suitable for assaying the phosphorylation state of any combination of 1, 2, 3, 4, 5, 6, 7, 8, 9, 10, or 11 of the mentioned markers and/or the total amount of COX-2. In one embodiment, the agents are suitable for assaying the phosphorylation state of AKT (e.g., S473), cABL (e.g., T735), ERK (e.g., T42/44), p38MAPK (e.g., T180-182), EGFR (e.g., Y992), and/or COX-2. The agent may be, e.g., an antibody (such as a monoclonal antibody) or other ligand specific for a particular phosphorylation isoform of one of the mentioned phosphoproteins, or for COX-2. Another aspect of the invention is a kit for predicting the prognosis of, or the likelihood of later developing metastasis in, or the desirability of administering an aggressive therapy to, a subject with colorectal cancer, comprising a collection of agents as discussed above, optionally packaged in one or more containers.

Another aspect of the invention is a pharmaceutical composition or kit for treating a subject in need thereof, comprising an effective amount of one or more of the inhibitory or stimulatory agents as discussed herein, or a combination thereof. For example, the pharmaceutical composition can comprise an effective amount of (a) an EGFR inhibitor and/or a cABL inhibitor and (b) a p38MAPK inhibitor, and/or an AKT inhibitor, and/or an ERK inhibitor. Another aspect of the invention is a pharmaceutical composition comprising an effective amount of carboxyamido imidazole (CAI) in combination with a pAKT inhibitor, a pcABL inhibitor, a pERK inhibitor, a COX-2 inhibitor, or a pEGFR inhibitor. In a pharmaceutical composition of the invention, the pEGFR inhibitor can be, e.g., TARCEVA, LAPATINIB, IRESSA, ERBITUX, and/or BEVTUZIMAB; the pABL inhibitor can be, e.g., GLEEVEC and/or SUTENT; and/or the COX-2 inhibitor can be, e.g., VIOXX and/or CELEBREX.

A pharmaceutical composition comprises a pharmaceutically acceptable carrier. In a kit, the inhibitory or stimulatory agent may be in a container. A pharmaceutical composition or kit of the invention may also comprise one or more conventional chemotherapeutic agents that can be administered in conjunction with the inhibitor(s) and/or activator(s).

The nucleotide and amino acid sequences of the above-mentioned genes and proteins are well-known and can be determined routinely, as well as downloaded from various known databases. See, e.g., the world wide web site, ncbi.nlm.nih.gov.

A “sample,” as used herein, can include any suitable cell or tissue that can be assayed to determine the phosphorylation state of one or more of the phosphoproteins therein, or the total amount of the COX-2 protein. Suitable samples include, e.g., peripheral blood cells, and biopsies of tumors, such as needle biopsies or gross surgical specimens procured upon primary tumor resectioning. A sample may be, e.g., fresh, frozen (e.g. flash frozen), or preserved in a manner that retains the protein content of the cell, including the levels of protein phsophorylation.

A “subject,” as used herein, includes any animal that has colorectal cancer. Suitable subjects (patients) include laboratory animals (such as mouse, rat, rabbit, or guinea pig), farm animals, and domestic animals or pets (such as a cat or dog). Non-human primates and, preferably, human patients, are included.

As used herein, a “significantly elevated” level of phosphorylation is a level whose difference from a negative reference standard is statistically significant, using statistical methods that are appropriate and well-known in the art, generally with a probability value of less than five percent chance of the change being due to random variation. For example, the phosphorylation of a residue in a diagnostic biomarker of the invention in a subject that has or is likely to have a metastatic form of colorectal cancer may range from 20% to more than 200% higher than the level observed in a subject who does not have cancer, or who has a form of colorectal cancer that is not metastatic.

A “significantly reduced” level of phosphorylation, as used herein, is a comparable difference from a positive reference standard, or from a subject that has a metastatic form of colorectal cancer. A significantly reduced level of phosphorylation is a level whose difference from a positive reference standard is statistically significant, using statistical methods that are appropriate and well-known in the art, generally with a probability value of less than five percent chance of the change being due to random variation. For example, the phosphorylation of a residue in a diagnostic biomarker of the invention in a subject that is unlikely to have a metastatic form of colorectal cancer may range from 20% to about 90% lower than the level observed in a subject who has a metastatic form of colorectal cancer (e.g., reduced to a level lower than about 80% of the positive reference standard, or as low as an undetectable amount).

The level of phosphorylation of a biomarker, as used herein, refers to the level of phosphorylation at a given amino acid residue of a protein (e.g., on the amino acid side chain). An increase in the amount of phosphorylation of a protein (e.g., an increase in the total amount per cell of a phosphoprotein isoform of interest) can reflect the total amount of phosphorylated protein or an increased frequency of phosphorylations at the amino acid residue. In general, the total amount of protein that is phosphorylated at the noted amino acid residue is measured, per sample or per cell in the sample.

In one embodimenit of the invention, the level of phosphorylation of a biomarker is determined by preparing positive and negative reference standards derived from tissue culture cells.

To generate a “positive” reference standard, one can first process cells obtained from a biopsy specimen (such as a human biopsy specimen) from a subject (or a pool of subjects) that is known to have a metastatic form of colorectal cancer. Protein extracts can be prepared from the tissue and the level of phosphorylation (or range of values) at the phospho-endpoints of interest determined as described herein. The median value of such samples can serve as a positive reference standard.

To generate a “negative” reference standard, one can process cells from a comparable tissue from a subject (or a pool of subjects) that is known not to have cancer (a “normal” subject), or to have a non-metastatic form of colorectal cancer. Protein extracts can be prepared from the tissue and the level of phosphorylation (or range of values) at the phospho-endpoints of interest determined as described herein. The median value of Such samples can serve as a negative reference standard.

In variations of the above method, the determination of the positive or negative standard may be based on published data, retrospective studies of tissues from patients who have had or been free of metastasis, and other information as would be apparent to a person of ordinary skill implementing the method of the invention.

However, using such tissue from subjects as a clinical diagnostic reference standard is generally not practical on a routine basis. Instead, it is preferable to generate negative and positive reference standards by using lysates from cells in culture, and establishing a cut-point value by a direct comparison of the cell culture lysates to a true positive (e.g. endpoint values derived from subjects with a metastatic form of colorectal cancer, as described above) and true negative (e.g. endpoint values derived from subjects that do not have metastatic forms of colorectal cancer, as described above). To accomplish this, one can first screen a variety of cells in culture, either primary cells or, preferably, cell lines (e.g., any of a variety of well-known cell lines for which treatment with a mitogen, such as EGF or pervandate, will induce phsophorylation).

These or other types of cells in culture can be propagated directly, under conventional conditions, so that the proteins of the invention are not phosphorylated or are phosphorylated to a minimal degree; or they can be incubated under conventional conditions with a suitable mitogen that will globally activate signaling networks, such as pervanadate, or a growth factor, such as epidermal growth factor (EGF).

Protein extracts are then prepared from the various cell lines, which have been incubated under the various conditions, using conventional procedures; and the level of phosphorylation at the phospho-endpoints of interest determined as described herein, and compared directly to the true positive and true negative clinical samples as a bridging experiment. In this way, one can establish conditions such that particular cells, cultured under particular defined conditions (stimulated or not), express an amount of phosphorylation of the phosphoprotein isoforms of the invention that is directly comparable to those of a subject that has, or does not have, a metastatic form of colorectal cancer. Utilizing the cut-point values derived from median values of known true clinical positives and negatives, and bridging these values to a cell line reference standard can then provide a “positive reference standard” or a “negative reference standard,” respectively. The positive and negative values may be selected using conventional statistical tools, so that values measured from a clinical sample that are higher than a negative reference standard value can be accepted as being predictive of metastasis, and measured values that are lower than a positive reference standard can be accepted as being predictive of no metastasis.

Alternatively, the level of phosphorylation in a purified sample of the analyte (e.g., one or more of the phosphorylated protein isoforms of the invention) of known concentration can be used.

For each protein whose level of phosphorylation is determined, the value can be normalized, e.g., to the total protein in the cell; or to the amount of a constitutively expressed protein (from a housekeeping gene), such as actin; or the amount of a phosphoprotein may be compared to the amount of its non-phosphorylated counterpart.

The level of phosphorylation of a given amino acid residue can be measured qualitatively or quantitatively. The amount (quantity) of phosphorylation at a given residue may be higher than is observed at the same residue in a control sample. That is, it may be hyperphosphorylated. In addition to hyperphosphorylation as a detection threshold, the presence or absence of phosphorylation at the noted residues can also be utilized. Alternatively, a qualitative scale (such as a scale of 1 to 5) can be used.

Methods for measuring the level of phosphorylation at an amino acid residue, and/or to determine the activation of a signaling pathway, are conventional and routine. In one embodiment, the measurement relies on the existence of sets of antibodies that are specific for either the non-phosphorylated or the phosphorylated forms of a particular amino acid residue of interest in the context of a protein of interest (such as a kinase substrate). Antibodies can be used, e.g., that are specific for non-phosphorylated or phosphorylated isoforms of the biomarkers of the invention. Such antibodies are commercially available or can be generated routinely, using conventional procedures. In one embodiment, a synthetic peptide comprising an amino acid of interest from a protein of interest (either in the non-phosphorylated or phosphorylated form) is used as an antigen to prepare a suitable antibody. The antibody can be polyclonal or monoclonal. A skilled worker will recognize a variety of suitable antibodies, antibody fragments or aptamers that can be used. Antibodies are selected and verified to detect only the phosphorylated version of the protein but not the non-phosphorylated version of the native or denatured protein, and vice-versa.

Such antibodies can be used in a variety of ways. For example, one can prepare whole cell lysates from patient samples and spot them in an array format onto a suitable substrate, such as nitrocellulose strips or glass slides. Preferably, the proteins in the samples are denatured before spotting. In general, the cells are spotted at serial dilutions, such as two-fold serial dilutions, to provide a wide dynamic range. Suitable controls, such as positive controls or controls for base line values, can be included. Each array is then probed with a suitable detectable antibody, as described above, to determine and/or to quantitate which amino acid residue(s) in the various proteins of interest are phosphorylated. Methods for immuno-quantitation are conventional. For a further discussion of this method of reverse phase protein lysate microarrays (RPMA), see, e.g., Nishizuka et al. (2003) Proc. Natl. Acad. Sci. 100,14229-14239.

Other suitable assays employing such antibodies to assess the level and/or degree of phosphorylation at a residue of interest include, e.g., colorimetric assays, immunoassays (such as immunohistochemistry, ELISAs, etc.), assays based on fluorescent readouts, Western blots, suspension bead assays, immunoprecipitation, mass spectroscopy, and other conventional assays. Suitable methods include those that can detect the phosphoprotein in a very small sample (e.g. about 200 cells). Alternatively, methods can be used that are suitable for a large sample size (e.g. about 20,000-25,000 cells).

Assays to measure the presence and/or amount of phosphorylated residues can be readily adapted to high throughput formats, e.g. using robotics.

Methods for determining the total amount of non-phosphorylated proteins, such as COX-2, are conventional, as are method for determining suitable positive and negative reference standards, and for determining if a significantly increased amount of the protein is present in a subject compared to a negative reference standard.

Suitable controls for assays of the invention will be evident to the skilled worker. For example, to provide for quality control, each set of proteins tested (e.g. in the form of a protein micro-array) may contain antigen controls, cell lysate controls, and/or a reference lysate. Each patient analyte sample can be normalized to total protein and quantitated in units relative to the reference “printed” on the same array. Each reference and control lysate can be printed in the same dilution series as patient samples and be immunostained at the same time, with identical reagents as the patient samples. All samples can be printed in duplicate in 4-point dilution curves.

To provide for quality assurance, samples can be processed and analyzed in real time, e.g. as they are received at a suitable processing facility that meets applicable regulatory standards. Samples may consist of Cytolyte preserved samples. A test set with matched fiozen samples can verify the adequacy of specimen preservation. Techniques can be carried out at room temperature. Samples may be obtained by core needle biopsy.

Following the determination of the level of phosphorylation of a marker protein by a method as discussed herein, the values can be reported, e.g. in the form of a panel or suite of values, to physicians to improve therapy decisions for their patients. With such a report, cancer and other diseases with a common diagnosis may be stratified at a molecular level, according to the therapies that are likely to be effective. This allows for optimal personalized patient therapies. Some suitable systems for reporting the data are described in co-pending provisional application, Ser. No. 60/935,106, filed Mar. 27, 2007. Such reports can provide a comprehensive list of the particular phosphoproteins in question, normal reference levels or ranges for each, and the measured level of phosphorylation of the protein in the patient sample.

One aspect of the invention is a method for treating a subject that has been determined by a method of the invention, to have a poor prognosis, to have a form of colorectal cancer that is likely to later metastasize, and/or to be a good subject for aggressive therapy and/or targeted therapy.

An inhibitor or activator of the invention can be administered when an aberrant total amount of phosphoprotein, or level of phosphorylation at a particular residue (or, in the case of COX-2, the total amount of protein) is observed in at least one of the 12 mentioned protein markers, in the sample obtained from the subject.

In one embodiment of the invention, the inhibitor is a kinase inhibitor that reduces the phosphorylation state of a phosphoprotein marker of the invention that is over-phosphorylated, or is an enzyme inhibitor that reduces the activity of a protein marker of the invention that is over-activated or over-expressed. Other suitable inhibitors include, for example, siRNAs directed against nucleic acids encoding an over-activated or over-expressed protein marker of the invention; and antibodies, e.g., polyclonal or monoclonal antibodies, aptamers or other ligands directed against a protein marker of the invention. In another embodiment, the activator is a kinase which increases the phosphorylation state of a phosphoprotein marker of the invention that is under-phosphorylated.

Examples of AKT-kinase (e.g., S473) (also known as protein kinase B) inhibitors include, but are not limited to, e.g.,
Akt-1-1 (inhibits Akt1) (Barnett el al. (2005) Biochem. J., 385 (Pt.2), 399-408);
Akt-1-1,2 (inhibits Ak1 and 2) (Barnett et al. (2005) Biochem. J. 385 (Pt.2), 399-408);
API-59CJ-Ome (e.g., Jin el al. (2004) Br. J. Cancer 91, 1808-12);
I-H-imidazo[4,5-c]pyridinyl compounds (e.g., WO05011700);
indole-3-carbinol and derivatives thereof (e.g., U.S. Pat. No. 6,656,963; Sarkar and Li (2004) J Nutr. 134(12 Suppl), 3493S-3498S);
perifosine (e.g., interferes with Akt membrane localization; Dasmahapatra et al. (2004) Clin. Cancer Res. 10(15), 5242-52, 2004);
phosphatidylinositol ether lipid analogues (e.g., Gills and Dennis (2004) Expert. Opin. Investig. Drugs 13, 787-97);
triciribine (TCN or API-2 or NCI identifier: NSC 154020; Yang et al. (2004) Cancer Res. 64, 4394-9).

Examples of pcABL (e.g., the T735 isoform) inhibitors include, but are not limited to, e.g., GLEVEC, SUTENT, and SKI-606 (Thaimattam et al. (2005) Bioorg Med Chem 13, 4704-12.

Examples of pERK (e.g., the T42/44 isform) inhibitors include, but are not limited to, e.g., the ERK inhibitor PD98059 and the ERK/MEK inhibitor U0126 (Zelivianski el al. (2003) Int. J Cancer 107, 478-85.

Examples of pMARCKS (e.g., the S152-156 isoform) inhibitors include, but are not limited to, e.g., the isoquinolinesulfonamide derivatives, H-1152, HA-1077, and Y-27632 (Ikenoya et al. (2002) J Neurochem 81, 9-16).

Examples of pp38MAPK (e.g., the T180-182 isoform) inhibitors include, but are not limited to, e.g., SB-239063 and SB 220025 (Legos et al. (2002) Eur J Pharmacol 447, 37-42).

Examples of pSTAT1 (e.g., the Y701 isoform) inhibitors include, but are not limited to, e.g., fludarabine (Terui et al. (2004) Biochem J 380, 203-209).

Examples of PTEN (e.g., the S380 isoform) inhibitors include, but are not limited to, Bisperoxovanadium compound.

Examples of pEGFR (e.g., the Y992 isoform) inhibitors include, but are not limited to, e.g., TARCEVA, IRESSA, LAPSTINIB. ERBITIX and BEVTUZIMAB.

Examples of COX-2 inhibitors include, but are not limited to, e.g., VIOXX and CELEBREX.

Assays or treatment methods related to the mentioned phosphoproteins in their unphosphorylated and phosphorylated states (or COX-2) can be used in accordance with the present invention, irrespective of the mechanism of action. Thus, although it is believed that the mechanism underlying metastasis may be affected by the phosphorylation state of one or more of the indicated markers, or by the amount of COX-2, the present invention is not bound to any mechanism by which the theraniostic, therapeutic, and/or prognostics methods achieve their success.

The inhibitors or activators discussed herein can be formulated into various compositions, e.g., pharmaceutical compositions, for use in therapeutic treatment methods. The pharmaceutical compositions can be assembled as a kit. Generally, a pharmaceutical composition of the invention comprises an antimetastatic-effective amount of the inhibitor. An “antimetastatic effective amount,” as used herein, is an amount that is sufficient to effect at least a detectable therapeutic response in the individual over a reasonable time frame. For example, it can ameliorate, at least to a detectable degree, the symptoms of metastasis, or can inhibit the spread of a tumor, etc.

The composition can comprise a carrier, such as a pharmaceutically acceptable carrier. By “pharmaceutically acceptable” is meant a material that is not biologically or otherwise undesirable, i.e., the material may be administered to a subject without causing any undesirable biological effects or interacting in a deleterious manner with any of the other components of the pharmaceutical composition in which it is contained. The carrier is selected to minimize any degradation of the active ingredient and to minimize any adverse side effects in the subject, as would be well known to one of skill in the art. For a discussion of pharmaceutically acceptable carriers and other components of pharmaceutical compositions, see, e.g., Remington's Pharmaceutical Sciences, 18th ed., Mack Publishing Company, 1990.

A pharmaceutical composition or kit of the invention can contain other pharmaceuticals (such as chemotherapeutic agents), in addition to the inhibitors or stimulators of the invention. The other chemotherapeutic agent(s) can be administered at any suitable time during the treatment of the patient, either concurrently or sequentially.

One skilled in the art will appreciate that the particular formulation will depend, in part, upon the particular inhibitory or stimulatory agent of the invention, or other chemotherapeutic agent, that is employed, and the chosen route of administration. Accordingly, there is a wide variety of suitable formulations of compositions of the present invention.

Among the conventional chemotherapeutic agents that can be administered to a subject in conjunction with one or more inhibitors of activators of the invention are the agents listed in Table 1.

TABLE 1

| Mechanism of action | Class_(drug names) |
|---|---|
| Alkylating agents | Nitrogen mustards: (Chlorambucil, Chlormethine, |
| | Cyclophosphamide, Ifosfamide, Melphalan). Nitrosoureas: |
| | (Carmustine, Fotemustine, Lomustine, Streptozocin). Platinum: |
| | (Carboplatin, Cisplatin, Oxaliplatin, BBR3464). Busulfan, |
| | Dacarbazine, Mechlorethamine, Procarbazine, Temozolomide, |
| | ThioTEPA, Uramustine |
| Antimetabolites: | Folic acid: (Methotrexate, Pemetrexed, Raltitrexed). Purine: |
| | (Cladribine, Clofarabine, Fludarabine, Mercaptopurine, |
| | Tioguanine). Pyrimidine: (Capecitabine). Cytarabine, |
| | Fluorouracil, Gemcitabine |
| Plant alkaloids: | Taxane: (Docetaxel, Paclitaxel). Vinca: (Vinblastine, |
| | Vincristine, Vindesine, Vinorelbine). |
| Cytotoxic/antitumor | Anthracycline family: (Daunorubicin, Doxorubicin, Epirubicin, |
| antibiotics: | Idarubicin, Mitoxantrone, Valrubicin). Bleomycin, Hydroxyurea, |
| | Mitomycin |
| Topoisomerase inhibitors: | Topotecan, Irinotecan, Podophyllum: (Etoposide, Teniposide). |
| Monoclonal antibodies: | Alemtuzumab, Bevacizumab, Cetuximab, Gemtuzumab, |
| | Panitumumab, Rituximab, Trastuzumab |
| Photosensitizers: | Aminolevulinic acid, Methyl aminolevulinate, Porfimer sodium, |
| | Verteporfin |
| Other: | Alitretinoin, Altretamine, Amsacrine, Anagrelide, Arsenic |
| | trioxide, Asparaginase, Bexarotene, Bortezomib, Celecoxib, |
| | Denileukin diftitox, Erlotinib, Estramustine, Gefitinib, |
| | Hydroxycarbamide, Imatinib, Pentostatin, Masoprocol, Mitotane, |
| | Pegaspargase, Tretinoin |
| Hormones | Tamoxafin, Progesterones |

Formulations suitable for oral administration can consist of liquid solutions, such as an effective amount of the agent dissolved in diluents, such as water, saline, or fruit juice; capsules, sachets or tablets, each containing a predetermined amount of the active ingredient, as solid, granules or freeze-dried cells; solutions or suspensions in an aqueous liquid; and oil-in-water emulsions or water-in-oil emulsions. Tablet forms can include one or more of lactose, mannitol, corn starch, potato starch, microcrystalline cellulose, acacia, gelatin, colloidal silicon dioxide, croscarmellose sodium, talc, magnesium stearate, stearic acid, and other excipients, colorants, diluents, buffering agents, moistening agents, preservatives, flavoring agents, and pharmacologically compatible carriers. Suitable formulations for oral delivery can also be incorporated into synthetic and natural polymeric microspheres, or other means to protect the agents of the present invention from degradation within the gastrointestinal tract.

Formlulations suitable for parenteral administration (e.g. intravenous) include aqueous and non-aqueous, isotonic sterile injection solutions, which can contain anti-oxidants, buffers, bacteriostats, and solutes that render the formlulation isotonic with the blood of the intended recipient, and aqueous and non-aqueous sterile suspensions that can include suspending agents, solubilizers, thickening agents, stabilizers, and preservatives. The formulations can be presented in unit-dose or multi-dose sealed containers, such as ampules and vials, and can be stored in a freeze-dried (lyophilized) condition requiring only the addition of the sterile liquid carrier, for example, water, for injections, immediately prior to use. Extemporaneous injection solutions and suspensions can be prepared from sterile powders, granules, and tablets of the kind previously described.

The inhibitory or stimulatory agents of the invention, alone or in combination with other chemotherapeutic agents, can be made into aerosol formulations to be administered via inhalation. These aerosol formulations can be placed into pressurized acceptable propellants, such as dichlorodifluoromethane, propane, nitrogen and the like.

One skilled in the art will appreciate that a suitable or appropriate formulation can be selected, adapted or developed based upon the particular application at hand.

Dosages for an inhibitory or stimulatory agent of the invention can be in unit dosage form, such as a tablet or capsule. The term “unit dosage form” as used herein refers to physically discrete units suitable as unitary dosages for human and animal subjects, each unit containing a predetermined quantity of an agent of the invention, alone or in combination with other chemotherapeutic agents, calculated in an amount sufficient to produce the desired effect in association with a pharmaceutically acceptable diluent, carrier, or vehicle.

One skilled in the art can easily determine the appropriate dose, schedule, and method of administration for the exact formulation of the composition being used, in order to achieve the desired anti-metastatic effective amount or effective concentration of the agent in the individual patient. One skilled in the art also can readily determine and use an appropriate indicator of the “effective concentration” of the compounds of the present invention by a direct or indirect analysis of appropriate patient samples (e.g., blood and/or tissues).

The dose of an inhibitory or stimulatory agent of the invention, or composition thereof, administered to an animal, particularly a human, in the context of the present invention should be sufficient to effect at least a therapeutic response in the individual over a reasonable time frame (an anti-metastatic effective amount). The exact amount of the dose will vary from subject to subject, depending on the species, age, weight and general condition of the subject, the severity or mechanism of any disorder being treated, the particular agent or vehicle used, its mode of administration and the like. The dose used to achieve a desired antimetastatic concentration in vivo will be determined by the potency of the particular inhibitory agent employed, the pharmacodynamics associated with the agent in the host, the severity of the disease state of infected individuals, as well as, in the case of systemic administration, the body weight and age of the individual. The size of the dose also will be determined by the existence of any adverse side effects that may accompany the particular inhibitory agent, or composition thereof, employed. It is generally desirable, whenever possible, to keep adverse side effects to a minimum.

When given in combined therapy, the other (conventional) chemotherapeutic agent, for example, can be given at the same time as the inhibitor or activator, or the dosing can be staggered as desired. The two (or more) drugs also can be combined in a composition. Doses of each can be less when used in combination than when either is used alone.

Another embodiment of the invention is a kit useful for any of the methods disclosed herein (e.g. for a diagnostic or therapeutic method); such a kit can comprise one or more of the inhibitors or activators, or diagnostic reagents, discussed herein. For example, a kit suitable for therapeutic treatment of a metastatic cancer in a subject may further comprise a pharmaceutically acceptable carrier and, optionally, a container or packaging material. A diagnostic kit can contain suitable agents for determining the phosphorylation state (or, in the case of COX-2, the total amount) of a marker of the invention. The agents can be, e.g., antibodies, including polyclonal or monoclonal antibodies, aptamers, or other ligands that bind specifically to the protein of interest (e.g., in the case of a phosphoprotein, that bind specifically to a phosphorylated isoform of interest). Among other uses, kits of the invention can be used in experimental applications. A skilled worker will recognize components of kits suitable for carrying out any of the methods of the invention.

Optionally, the kits comprise instructions for performing the method. Optional elements of a kit of the invention include suitable buffers, pharmaceutically acceptable carriers, or the like, containers, or packaging materials. The reagents of the kit may be in containers in which the reagents are stable, e.g., in lyophilized form or stabilized liquids. The reagents may also be in single use form, e.g., in single dosage form.

In the foregoing and in the following examples, all temperatures are set forth in uncorrected degrees Celsius; and, unless otherwise indicated, all parts and percentages are by weight.

### EXAMPLES

### Example I

### Materials and Methods

1. Reverse Phase Protein Microarrays. Microdissected cells, generated by previously published methods (e.g. Petricoin el al. (2005), J. Clin Oncol 23, 3614-3621; Liotta et al. (2003) Cancer Cell 3, 317-325; Sheehan el al. (2005) Mol Cell Proteomics 4, 346-365) were subjected to lysis and reverse phase protein microarrays were printed in duplicate with the whole cell protein lysates as described by Sheehan el al.(2005), supra. Briefly, the lysates were printed on glass backed nitrocellulose array slides (FAST Slides Whatman, Florham Park, N.J.) using a GMS 417 arrayer (Affymetrix, Santa Clara, Calif.) equipped with 500 μm pins. Each lysate was printed in a dilution curve representing neat, 1:2, 1:4, 1:8, 1:16 and negative control dilutions. The slides were stored with desiccant (Drierite, W. A. Hammond, Xenia, Ohio) at −20° C. prior to immunostaining.

2. Bioinformatics method for microarray analysis. Each array was scanned, spot intensity analyzed, data normalized, and a standardized, single data value was generated for each sample on the array (Image Quanit v5.2, GE Healthcare, Piscataway, N.J.). Spot intensity was integrated over a fixed area. Local area background intensity was calculated for each spot with the unprinted adjacent slide background. This resulted in a single data point for each sample, for comparison to every other spot on the array. Wilcoxon two-sample rank sum test was used to compare values between two groups. P values less than 0.05 were considered significant.

### Example II

Identification of Signal Pathway Alterations and Drug Targets that can Distinguish Colorectal Cancer that Metastasizes from Colorectal Cancer that does not

A study set was used of colorectal carcinoma that had presented with hepatic metastasis and colorectal carcinomas taken from human subjects at surgery that had no evidence of metastasis, and upon follow up, did not present with metastasis. The surgical samples were processed with laser capture microdissection and pure cancer cell populations were lysed and subjected to reverse phase protein microarray analysis. Using this technique, we were able to measure the phosphorylation state of 70 kinase substrates. Molecular network analysis was performed using commercially available software (Microvigene, VigeneTech, MA). Of the 70 phosphoendpoints analyzed, 12 were statistically significantly (via Student t-test p<0.05) expressed between the metastatic (aggressive) vs non-metastatic (indolent) cancers. These results are shown in FIG. 1. Of these 12 phosphoendpoints, 10 were elevated in the patients that presented with metastasis vs those without mets and 3 were elevated in the patients with non-metastatic disease. Unexpectedly, the some of these phosphorylation represent drug targets already in use in specific targeted therapies.
Elevation of cAbl (T245)=GLEEVEC
Elevation of COX-2=VIOXX
Elevation of pEGFR=TARCEVA

Moreover, many of these endpoints, such as ERK, AKT, and p38 are targets for other molecular inhibitors that are being developed. In addition, as a panel of markers, these endpoints can represent a theranostic opportunity that can distinguish aggressive from non-aggressive disease, good prognosis from bad prognosis, and provide a basis for chemopreventative or proactive therapy with COX-2, egfr, abl or other kinase directed therapies.

Some of the tested phosphoendpoints that were not significantly correlated with metastasis are shown below in Table 2:

TABLE 2

| Variable | P Value |
|---|---|
| cErb2-HER2 | 0.1322 |
| Cl Caspase3 D175 | 0.425 |
| COX2 | 0.0845 |
| Estrogen rec alfa | 0.7128 |
| FKHR S256 | 0.3821 |
| p4EBP1 T70 | 0.5351 |
| pAdducin S662 | 0.3882 |
| pASK1 S83 | 0.2865 |
| pBAD S112 | 0.0952 |
| pBcl2 S70 | 0.6058 |
| pCATENIN b | 0.0714 |
| pChk2 S33/35 | 0.2629 |
| pcKit Y719 | 0.7141 |
| pcRaf S338 | 0.0734 |
| pCREB S133 | 0.887 |
| pEGFR Y1173 | 0.2029 |
| pEGFR Y992 | 0.7292 |
| peNOS-NOSIII S116 | 0.7907 |
| pErbB2 Y1248 | 0.3866 |
| pERK T202-204 | 0.021 |
| pEstrogen rec (S118) | 0.8203 |
| pFAK Y397 | 0.8027 |
| pGSK3a-8 Y279 -216 | 0.5112 |
| pIKBa S32 | 0.0508 |
| pIRS1 S612 | 0.5843 |
| pJak1 Y1022-1023 | 0.063 |
| pMARCKS S152-156 | 0.1586 |
| pMEK1/2 S217/221 | 0.4788 |
| pMSK1 S360 | 0.3736 |
| pmTOR S2248 | 0.0652 |
| pNFkB S563 | 0.6608 |
| pp38 MAPK T180-182 | 0.0613 |
| pP70S6 T389 | 0.1761 |
| pP90RSK S380 | 0.0812 |
| pPAK1-PAK2 S119/204 | 0.0878 |
| S192/197 | |
| pPKAC T197 | 0.0584 |
| pPKC delta T505 | 0.5729 |
| pRas GRF1 S916 | 0.5456 |
| pSAPK-JNK T183-185 | 0.2499 |
| pSTAT1 Y701 | 0.3458 |
| pSTAT3 S727 | 0.1429 |
| pVEGFR Y996 | 0.0678 |
| S6 PROT RIB S235-236 | 0.376 |
| Smac Diablo | 0.3633 |

### Example III

Studies in Animal Models of Colorectal Cancer Showing that an Inhibitor of a Target of the Invention can Inhibit Metastasis

Causal significance of the signaling activation status as an underpinning cause of the metastatic process and thereby a therapeutic target for prevention of future metastasis in patients that present with colorectal cancer without metastasis is tested in animal model systems.

In a first animal model system, the rat BDIX strain is injected with syngenieic colorectal DHD-K12 cell line cells into the splenic vein; the injected cells will quickly form liver metastasis in 15 days and lung metastasis in 20 days. The rats are pretreated with the following kinase inhibitors, either alone or in combination: an EGFR inhibitor; an AKT inhibitor; a COX-2 inhibitor; an ERK inhibitor; a p38 inhibitor; a PKC inhibitor; a cABL inhibitor; a STAT1 inhibitor, using inhibitors as discussed herein.

In a second animal model system, the inhibitors are given concurrently to the rats with the splenic injection.

It is expected that the inhibitors will inhibit the formation of metastatic colonies, confirming that the activity of these phosphoprotein enzymes are necessary and sufficient for the formation of metastasis, and providing mechanistic evidence that these proteins in the egfr and cabl growth factor pathway through erk and akt activation are good candidates for both prognostic determination as well as targets for therapy for prevention.

From the foregoing description, one skilled in the art can easily ascertain the essential characteristics of this invention, and without departing from the spirit and scope thereof, can make changes and modifications of the invention to adapt it to various usage and conditions and to utilize the present invention to its fullest extent. The preceding preferred specific embodiments are to be construed as merely illustrative, and not limiting of the scope of the invention in any way whatsoever. The entire disclosure of all applications, patents, and publications cited above, including U.S. Provisional Application No.60/854,724, filed Oct. 27, 2006, and in the figures are hereby incorporated in their entirety by reference.

1-32. (canceled)

33. A method for predicting if a subject with a colorectal carcinoma has a form of colorectal carcinoma that is likely to metastasize, comprising the steps of:
(a) analyzing a sample of the colorectal carcinoma from the subject to identify the presence of at least one of the following proteins:
a. AKT,
b. BAD,
c. cABL,
d. ERK,
e. MARCKS,
f. p38MAPK,
g. STAT1,
h. PTEN,
i. EGFR,
j. PAK1-PAK2,
k. PKC zeta/lambda, and
l. COX-2;
(b) measuring the phosphorylation state of any of the proteins a.-k. identified by step (a);
(c) measuring the total amount of COX-2 protein, if identified by step (a); and
(d) comparing the measurements provided by steps (b) and (c) to positive and/or negative reference standards, wherein:
an elevated phosphorylation state of at least one of proteins a.-i. compared to a negative reference standard for said proteins and/or an elevated total amount of COX-2 protein compared to a negative reference standard for said protein; and/or
a phosphorylation state of at least one of proteins a.-i. that is the same as a positive reference standard for said proteins, and/or a total amount of COX-2 protein that is the same as a positive reference standard for said protein; and/or
a reduced phosphorylation state of at least one of proteins j. and k. compared to a positive reference standard for said proteins, or a phosphorylation state of at least one of proteins j. and k. that is the same as a negative reference standard for said proteins,
indicates that the subject has a form of colorectal carcinoma that is likely to metastasize.

34. The method of claim 33, wherein the subject is a human patient and the colorectal carcinoma is likely to metastasize to the patient's liver.

35. The method of claim 34, wherein the step of analyzing the sample to identify the presence of at least one of the proteins comprises:
isolating epithelial cells from the sample;
lysing the epithelial cells; and
analyzing the lysate.

36. The method of claim 34, wherein the protein or proteins identified and measured are at least one of AKT, BAD, cABL, EGFR, PTEN, and COX-2.

37. The method of claim 34, wherein the protein or proteins identified and measured are at least one of BAD, EGFR, and COX-2.

38. A method for delaying or preventing metastasis in a human patient with a colorectal carcinoma, wherein:
if a sample of the colorectal carcinoma from the patient exhibits an elevated phosphorylation state of at least one of AKT, BAD, cABL, ERK, MARCKS, p38MAPK, STAT1, PTEN, and EGFR compared to a negative reference standard for said proteins, and/or a phosphorylation state of at least one of AKT, BAD, cABL, ERK, MARCKS, p38MAPK, STAT1, PTEN, and EGFR that is the same as a positive reference standard for said proteins, then treating the patient with an effective amount of an inhibitor of phosphorylation of at least one of the proteins identified by comparison to the reference standard; and/or
if the sample exhibits an elevated total amount of COX-2 protein compared to a negative reference standard for said protein and/or exhibits a total amount of COX-2 protein that is the same as a positive reference standard for said protein, then treating the patient with an effective amount of an inhibitor of COX-2 protein; and/or
if the sample exhibits a reduced phosphorylation state of at least one of PAK1-PAK2 and PKC zeta/lambda compared to a positive reference standard for said proteins, and/or a phosphorylation state of at least one of PAK1-PAK2 and PKC zeta/lambda that is the same as a negative reference standard for said proteins, then treating the patient with an effective amount of an activator of phosphorylation of at least one of the proteins identified by comparison to the reference standard.

39. The method of claim 38, wherein, if the sample exhibits an elevated phosphorylation state of at least one of AKT, BAD, cABL, EGFR, and PTEN compared to a negative reference standard for said proteins, and/or a phosphorylation state of at least one of AKT, BAD, cABL, EGFR, and PTEN that is the same as a positive reference standard for said proteins, then treating the patient with an effective amount of an inhibitor of phosphorylation of at least one of the proteins identified by comparison to the reference standard; and/or if the sample exhibits an elevated total amount of COX-2 protein compared to a negative reference standard for said protein and/or exhibits a total amount of COX-2 protein that is the same as a positive reference standard for said protein, then treating the patient with an effective amount of an inhibitor of COX-2.

40. The method of claim 38, wherein, if the sample exhibits an elevated phosphorylation state of at least one of BAD and EGFR compared to a negative reference standard for said proteins, and/or a phosphorylation state of at least one of BAD and EGFR that is the same as a positive reference standard for said proteins, then treating the patient with an effective amount of an inhibitor of phosphorylation of at least one of the proteins identified by comparison to the reference standard; and/or if the sample exhibits an elevated total amount of COX-2 protein compared to a negative reference standard for said protein and/or exhibits a total amount of COX-2 protein that is the same as a positive reference standard for said protein, then treating the patient with an effective amount of an inhibitor of COX-2.

41. The method of claim 39, wherein the AKT phosphorylation inhibitor is VQD-002 and/or Enzastaurin; the cABL phosphorylation inhibitor is imatinib, dasatinib, and/or sunitinib; the EGFR phosphorylation inhibitor is erlotinib, lapatinib, genfitinib, and/or cetuximab; the PTEN phosphorylation inhibitor is bisperoxovanadium; and the COX-2 inhibitor is rofecoxib and/or celecoxib.

42. A kit comprising agents for assaying the phosphorylation state of one or more of AKT, BAD, cABL, ERK, MARCKS, p38MAPK, STAT1, PTEN, EGFR, PAK1-PAK2, PCK zeta/lambda and for assaying the total amount of COX-2 in a colorectal carcinoma sample from a subject with colorectal carcinoma.

43. A kit comprising agents for assaying the phosphorylation state of one or more of AKT, BAD, cABL, PTEN, EGFR and for assaying the total amount of COX-2 in a colorectal carcinoma sample from a subject with colorectal carcinoma.

44. A kit comprising agents for assaying the phosphorylation state of BAD and/or EGFR and for assaying the total amount of COX-2 in a colorectal carcinoma sample from a subject with colorectal carcinoma.

45. The kit of claim 42, wherein the agents are antibodies.

46. The kit of claim 45, wherein the antibodies are monoclonal antibodies.

47. A pharmaceutical composition, comprising an effective amount of:
(a) an inhibitor of at least one of pAKT, pBAD, pcABL, pERK, pMARCKS, p38MAPK, pSTAT1, pPTEN, pEGFR, and COX-2; and
(b) an activator of at least one of pPAK1-PAK2 and pPKC zeta/lambda; and
(c) a pharmaceutically acceptable carrier.

48. A pharmaceutical composition, comprising an effective amount of:
(a) an inhibitor of two or more of pAKT, pBAD, pcABL, pERK, pMARCKS, p38MAPK, pSTAT1, pPTEN, pEGFR, and COX-2; and
(b) a pharmaceutically acceptable carrier.

49. A pharmaceutical composition, comprising an effective amount of:
(a) an inhibitor of two or more of pAKT, pBAD, pcABL, pPTEN, pEGFR, and COX-2, and
(b) a pharmaceutically acceptable carrier.

50. The pharmaceutical composition of claim 49, wherein the composition comprises an effective amount of an inhibitor of two or more of pBAD, pEGFR, and COX-2.

51. The pharmaceutical composition of claim 49 further comprising an effective amount of carboxyamido imidazole.

52. The pharmaceutical composition of claim 49, wherein the AKT phosphorylation inhibitor is VQD-002 and/or Enzastaurin; the cABL phosphorylation inhibitor is imatinib, dasatinib, and/or sunitinib; the EGFR phosphorylation inhibitor is erlotinib, lapatinib, genfitinib, and or cetuximab; the PTEN phosphorylation inhibitor is bisperoxovanadium; and the COX-2 inhibitor is rofecoxib and/or celecoxib.
